(19) United States
(12) Patent Application Publication
(10) Pub. No.: US 2026/0053371 A1
(43) Pub. Date: Feb. 26, 2026

(54) DETECTION OF SUBCUTANEOUS STRUCTURES USING OPTICAL AND PHOTOACOUSTIC SENSING

(71) Applicant: NuVue Corporation, Tucson, AZ (US)

(72) Inventor: Jeffrey H. Owen, Tucson, AZ (US)

(21) Appl. No.: 19/279,347

(22) Filed: Jul. 24, 2025

Related U.S. Application Data
(60) Provisional application No. 63/674,867, filed on Jul. 24, 2024.

(51) Int. Cl. A61B 5/00

(52) U.S. Cl. CPC A61B 5/0095; A61B 5/0075

(57) ABSTRACT

One or more sensor systems are used to detect subcutaneous structures (e.g., arteries, veins, nerves, hard tissue/bone, foreign objects, etc.) using a combination of optical (e.g., functional Near Infrared Spectroscopy (fNIRS)) and photoacoustic imaging (PAI), e.g., by placing LEDs of different wavelengths on the skin and processing the optical and photoacoustic signals produced therefrom. Certain embodiments will provide real-time imaging of subcutaneous structures such as for use during real-time diagnostics and surgery.

### CROSS-REFERENCE TO RELATED APPLICATION(S)

This patent application claims the benefit of U.S. Provisional Patent Application No. 63/674,867 entitled DETECTION OF SUBCUTANEOUS STRUCTURES USING OPTICAL AND PHOTOACOUSTIC SENSING filed Jul. 24, 2024, which is hereby incorporated herein by reference in its entirety.

### FIELD OF THE INVENTION

The invention generally relates to detection of subcutaneous structures using a combination of optical and photoacoustic sensing.

### BACKGROUND OF THE INVENTION

According to Wikipedia, photoacoustic spectroscopy is the measurement of the effect of absorbed electromagnetic energy (particularly of light) on matter by means of acoustic detection. Generally speaking, the absorbed energy from the light causes local heating, generating a thermal expansion which creates a pressure wave or sound. A photoacoustic spectrum of a sample can be recorded by measuring the sound at different wavelengths of the light. This spectrum can be used to identify the absorbing components of the sample. The photoacoustic effect can be used to study solids, liquids and gases.

### SUMMARY OF VARIOUS EMBODIMENTS

In accordance with one embodiment of the invention, a sensor system includes at least one optical receiver, at least one acoustic receiver, and a plurality of sets of LEDs, wherein each set of LEDs contains a distinct color of LED, and wherein the sensor system is configured to direct light from the LEDs into skin to induce both optical signals that are detected by the at least one optical receiver and photoacoustic signals that are detected by the at least one acoustic receiver to detect subcutaneous structures through a combination of optical and photoacoustic sensing.

In various alternative embodiments, each set of LEDs may include one, two, three, or more LEDs. The LEDs may be arranged in various configurations such as around the ultrasonic transducer. The plurality of sets of LEDs may include a set of orange LEDs, a set of UV LEDs, a set of red LEDs, a set of near-infrared LEDs, and/or a set of infrared LEDs. The at least one optical receiver may include at least one photodiode. The at least one acoustic receiver may include at least one ultrasonic receiver or transducer. The sensor system may include a controller configured to control the LEDs and/or process received optical and acoustic signals.

Certain embodiments may include an apparatus comprising at least one sensor system of the types summarized above. The apparatus may be provided in the form of a handheld wand, pad, or other form factor. The apparatus may include a plurality of sensor systems, which, for example, may be arranged in an array such as a 4×4 array. The apparatus may include other components such as a controller separate from the at least one sensor system configured to control the LEDs and/or process received optical and acoustic signals, an interface to an external controller, an internal power source (which may be rechargeable), and/or an interface to an external power source (which may be used to power the apparatus or recharge an internal power source).

Certain embodiments may include an apparatus of the types summarized above and a controller separate from the apparatus. The apparatus and the controller may communicate over a wired or wireless communication connection. The controller may provide power to the apparatus. The controller may include one or more processors, which may include a digital signal processor. The controller may be configured to control the LEDs and/or process received optical and acoustic signals. The controller may be configured to display detected subcutaneous structures on a display in real-time. The displayed subcutaneous structures may be color-coded and/or displayed in 3D and/or manipulable in 3D.

Embodiments also may include a method for detecting subcutaneous structures involving activating at least one LED to direct LED light into skin of a patient, receiving optical signals returning through the skin in response to the LED light, receiving photoacoustic signals returning through the skin in response to the LED light, and processing the optical signals and the photoacoustic signals to detect at least one subcutaneous structure. The at least one subcutaneous structure may include at least one of arteries, veins, nerves, hard tissue/bone, foreign objects, etc. The at least one LED may include LEDs of at least two different colors. The method may involve controlling the intensity of the LED light.

Additional embodiments may be disclosed and claimed.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 is a schematic diagram showing one embodiment in the form of a handheld wand including a single sensor system, in accordance with certain embodiments.

FIG. 2 is a schematic diagram showing one embodiment in the form of a pad including multiple sensor systems in an array configuration (in this example, a 4×4 array), in accordance with certain embodiments.

FIG. 3 is a schematic diagram showing a system in accordance with certain embodiments.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

Certain embodiments utilize one or more sensor systems to detect subcutaneous structures (e.g., arteries, veins, nerves, hard tissue/bone, foreign objects, etc.) using a combination of optical (e.g., functional Near Infrared Spectroscopy (fNIRS)) and photoacoustic imaging (PAI), e.g., placing LEDs of different wavelengths on the skin and processing the optical and photoacoustic signals produced therefrom. Certain embodiments will provide real-time imaging of subcutaneous structures such as for use during real-time diagnostics and surgery. For purposes of this disclosure and any accompanying claims, the term “set” includes one or more members, even if the set description is presented in the plural (e.g., a set of Xs can include one or more X).

In certain embodiments, to elicit the fNIRS and PAI data, LEDs of different wavelengths will be included in a device (e.g., embedded in a silicone or other substrate) that also includes one or more optical receivers (e.g., photodiodes), one or more acoustic receivers (e.g., ultrasonic receiver or transducer), and other components (e.g., miniature amplifiers, LED control circuitry, processor, communication technology such as Bluetooth or WiFi to transmit data to a computer, etc.). The size of the device and the number of LEDs can be varied, for example, based on the size of the image required.

In certain embodiments, to elicit fNIRS data, LEDs (e.g., red, near-infrared, and/or infrared) will shine light into the tissue under the silicone pad. The reflected energy will then be recorded by the optical receiver(s), e.g., photodiode(s), and processed to differentiate between arteries, veins, hard tissue, etc.

The light energy produced by the LEDs used for fNIRS (and optionally also light from other LEDs, e.g., ultraviolet LEDS and other color LEDs such as orange LEDs) will be used to elicit PAI data. Unlike fNIRS, which records reflected light energy, PAI consists of recording high frequency sound produced by the low intensity and instantaneous heating of structures under the device. This heating produces oscillations in the tissue, which in turn produces a high frequency sound wave. This sound wave is recorded by the acoustic receiver(s) to detect and image subcutaneous structures, and the subcutaneous structures can be displayed as a “motion picture” of underlying tissue in real-time. The data from the fNIRS optical receiver(s) and the PAI acoustic receiver(s) also can be displayed on a monitor and can be used to determine the status of these structures. In certain embodiments, the system will color code detected subcutaneous structures, e.g., arteries red, veins blue, and bone white, nerves green, metal silver, etc. Such imaging can be provided in 3D, e.g., to show not only the subcutaneous structures but also the relative depths of the structures.

Certain embodiments are expected to be particularly useful during surgical procedures. During surgical procedures, it is often necessary for the surgeon to introduce metal instruments (e.g., trocars, screws, etc.) into the surgical field in a blind fashion. For example, during spine surgery, the surgeon will want to place screws into the vertebra of the spine but cannot see the location of any nerves or vessels. If the metal instrument were to hit one of these vessels the patient could have surgically induced postoperative neurological deficits. With the disclosed devices, the surgeon can use the imaging pad to watch the introduction of metal instruments into the blind field. Once introduced, the surgeon will be able to move a cursor on a monitor to the tip of the instrument and push a button and also identify the location of these structures using the same method. This will provide the surgeon with information regarding orientation and distance between the instrument and these structures. As the instrument moves, these distances will adjust accordingly. This will allow the surgeon to avoid hitting the structures.

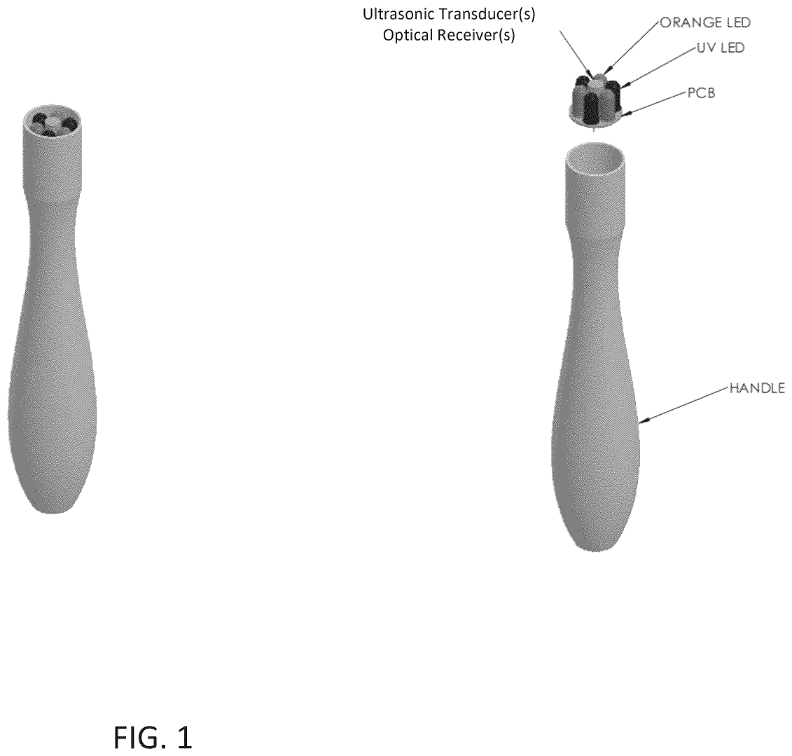

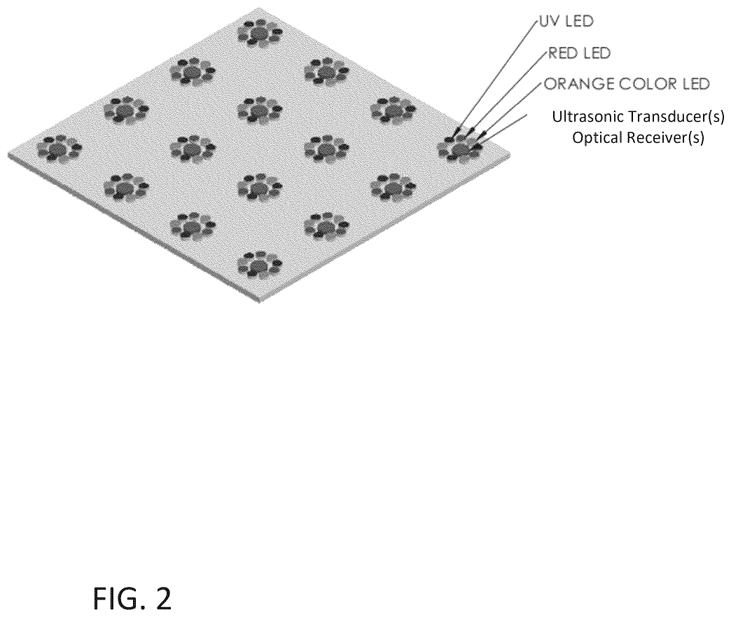

FIG. 1 is a schematic diagram showing one embodiment in the form of a handheld wand (which may be referred to as a “probe”) including a single sensor system, in accordance with certain embodiments. Here, the sensor system includes an acoustic receiver (e.g., ultrasonic transducer) surrounded by a set of orange LEDs and a set of ultraviolet (UV) LEDs (in this example, three of each type of LED) on a printed circuit board (PCB) within a handheld wand having a handle. It should be noted that the system could include more than one acoustic receiver (e.g., to enhance reception of acoustic signals or with different acoustic receivers tuned for specific frequencies), and the acoustic receiver(s) can be placed virtually anywhere within the system (e.g., inside of the LEDs as shown in FIG. 1, outside of the LEDs, or elsewhere). The system also includes one or more optical receivers (e.g., photodiodes), which could be placed virtually anywhere within the system (e.g., inside of the LEDs similar to the acoustic receiver, outside of the LEDs, or elsewhere). The sensor system generally would be placed on or dragged over the skin to detect subcutaneous structures by directing LED light into the skin to excite tissues to emit photoacoustic signals (e.g., sound waves) and receiving and processing photoacoustic signals induced by the LED light. The orange LEDs can be turned on to induce a first type of photoacoustic signal that can be received and processed by the ultrasonic transducer. The UV LEDs can be turned on to induce a second type of photoacoustic signal that can be received and processed by the ultrasonic transducer. For example, the handheld wand may include or be used with an internal or external controller and power source (e.g., internal battery which may be rechargeable, external power connection, etc.) that automatically alternates activation of the orange LEDs and the UV LEDs in any desired timing or sequence. Conceivably, both the orange LEDs and the UV LEDs could be activated simultaneously to induce a third type of photoacoustic signal that can be received and processed by the ultrasonic transducer. Thus, for example, the controller could automatically alternate activation of the orange LEDs, the UV LEDs, and both the orange and the UV LEDs in any desired timing or sequence. The internal or external controller could include a manual control, in addition to or in lieu of automatic control, allowing the user to manually turn activate selected LEDs or combinations of LEDs (e.g., to allow the user to do a first scan using orange LEDs, a second scan using UV LEDs, and possibly a third scan using both orange and UV LEDs. The controller of the handheld wand could process signals from the ultrasonic transducer itself and/or using an external processor (e.g., in communication with the handheld wand via a wired or wireless communication connection), e.g., to detect a nerve, blood vessel, bone, or other structure lying beneath the probe. Outputs from the controller or external processor may be displayed to the user such as on a tablet computer, smartphone, or other display device. It should be noted that the handheld wand could include multiple sensor systems and could be in other form factors including a pad configuration as in FIG. 2.

FIG. 2 is a schematic diagram showing one embodiment in the form of a pad including multiple sensor systems in an array configuration (in this example, a 4×4 array), in accordance with certain embodiments. Here, each sensor system includes an acoustic receiver (e.g., ultrasonic transducer) surrounded by a set of orange LEDs, a set of UV LEDs, and a set of red LEDs (in this example, three of each type of LED). It should be noted that the system could include more than one acoustic receiver (e.g., to enhance reception of acoustic signals or with different acoustic receivers tuned for specific frequencies), and the acoustic receiver(s) can be placed virtually anywhere within the system (e.g., inside of the LEDs as shown in FIG. 2, outside of the LEDs, or elsewhere). The system also includes one or more optical receivers (e.g., photodiodes), which could be placed virtually anywhere within the system (e.g., inside of the LEDs similar to the acoustic receiver, outside of the LEDs, or elsewhere). The sensor system generally would be placed on or dragged over the skin to detect subcutaneous structures by directing LED light into the skin and receiving and processing photoacoustic signals induced by the LED light. The orange LEDs can be turned on to induce a first type of photoacoustic signal that can be received and processed by the ultrasonic transducer. The UV LEDs can be turned on to induce a second type of photoacoustic signal that can be received and processed by the ultrasonic transducer. The red LEDs can be turned on to induce a third type of photoacoustic signal that can be received and processed by the ultrasonic transducer. For example, the pad may include or be used with an internal or external controller and power source (e.g., internal battery which may be rechargeable, external power connection, etc.) that automatically alternates activation of the orange LEDs, the UV LEDs, and the red LEDs in any desired timing or sequence. Conceivably, any combination of the orange LEDs, the UV LEDs, and the red LEDs could be activated simultaneously to induce additional types of photoacoustic signals that can be received and processed by the ultrasonic transducer. Thus, for example, the controller could activate orange/UV LEDs simultaneously, orange/red LEDs simultaneously, UV/red LEDs simultaneously, and/or all three simultaneously and could automatically alternate activation of individual and/or combinations of LEDs in any desired timing or sequence. The internal or external controller could include a manual control, in addition to or in lieu of automatic control, allowing the user to manually turn activate selected LEDs or combinations of LEDs (e.g., to allow the user to do a first scan using orange LEDs, a second scan using UV LEDs, a third scan using red LEDs, and possibly additional scans using one or more combinations of LEDs. The controller of the pad could process signals from the ultrasonic transducer itself and/or using an external processor (e.g., in communication with the handheld wand via a wired or wireless communication connection), e.g., to detect a nerve, blood vessel, bone, or other structure lying beneath the pad. Outputs from the controller or external processor may be displayed to the user such as on a tablet computer, smartphone, or other display device. It should be noted that the pad could include a single sensor system and could be in other form factors including a handheld wand as in FIG. 1.

It should be noted that embodiments of the present invention are not limited to any particular types, numbers, colors, or wavelengths of LEDs. Thus, for example, embodiments could include other color LEDs in addition to, or in lieu of, orange, red, and/or UV LEDs (e.g., infrared LEDs, near-infrared LEDs, etc.), could include more or fewer than three of a particular color LED, etc.

Embodiments also are not limited to any particular configuration of sensor systems. For example, the two-color system shown in FIG. 1 could be used in the pad of FIG. 2, and the three-color system shown in FIG. 2 could be used in the handheld wand of FIG. 1. Also, embodiments could include one or more ultrasonic transducers that are surrounded or otherwise associated with only a single-color LED, e.g., one sensor system having only orange LEDs, one sensor system having only UV LEDs, one sensor system having only red LEDs, etc.

In any of the described embodiments, the controller could control not only whether a particular LED or group of LEDs is on/off but also the intensity of the emitted LED light (e.g., by controlling the voltages provided the LEDs). Among other things, controlling LED light intensity could assist with determination of the depth of subcutaneous structures, e.g., if a particular structure is not detected at a first LED power level that penetrates to a first depth but is detected at a greater LED power level that penetrates to a greater depth, then the system might infer the depth of the structure based at least in part on the input voltage or other LED control. Thus, for example, the system could allow for complex “sweeps” of the skin such as by varying the LED color(s) and intensities. Controlling LED light intensity also could assist with calibration of the system, e.g., so that different color LEDs will penetrate the skin to the same depth

As discussed herein, embodiments additional could include one or more optical sensors to detect LED light returning back through the skin. Thus, certain embodiments can utilize a combination of optical and photoacoustic detection of subcutaneous structures in which optical and photoacoustic sensing could be used individually and/or in combination. Such embodiments could include a barrier to prevent LED light from reaching the optical sensor(s) directly, i.e., so that the optical sensor(s) only receive LED light returning back through the skin.

As discussed above, the system could use a controller external to the handheld wand or pad to process photoacoustic sensor signals (and also acoustic sensor signals in embodiments equipped with acoustic sensors). Such a controller also could control the LEDs and other system operations, e.g., to coordinate generation of LED light signals and processing of photoacoustic and/or optical signals. Such a controller could include, without limitation, one or more processors that can be co-located and/or distributed (e.g., cloud-based). The controller can provide a user interface (e.g., a graphical user interface) through which a user can control system operation and/or receive output from the system (e.g., information about detected subcutaneous structures including depictions of detected subcutaneous structures displayed on any appropriate display device (e.g., desktop computer, laptop computer, tablet computer, smartphone, smart display, wearable device, etc.).

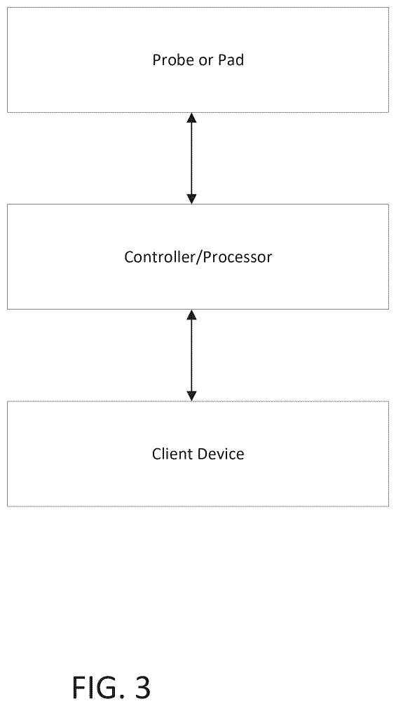

It should be noted that embodiments can include the sensor system, e.g., including LEDs, optical receiver(s), acoustic receiver(s), and other components. Embodiments also can include an apparatus including one or more sensor systems, e.g., in the form of a handheld wand, a pad, etc. Embodiments also can include a system comprising an apparatus and an external controller that can be configured to control the LEDs and/or process the received optical and acoustic signals. For example, as shown schematically in FIG. 3, a system can include a probe or pad that is coupled (e.g., via a wired or wireless communication system) to a controller or processor that controls the probe/pad and/or processes signals from the probe/pad. The system may allow a client device (e.g., a tablet computer, smartphone, or other client device) to interface with the controller/processor (e.g., via a wired or wireless communication system) such as to control the probe/pad, control the controller/processor, and/or view processed information such as 3D views of detected subcutaneous structures. Some controller/processor functions may be split between the probe/pad and an external controller/processor, e.g., with some processing done in the probe/pad (e.g., collection and transmission of received acoustic and/or optical signals) and some processing done by the external controller/processor (e.g., signal processing of the acoustic and/or optical signals to detect various types of subcutaneous structures).

Communication systems such as for coupling the probe/pad to the controller/processor and/or for coupling client devices to the controller/processor may be or include any network that carries data. Non-limiting examples of suitable networks that may be used in whole or in part as network include a private or non-private local area network (LAN), personal area network (PAN), storage area network (SAN), backbone network, global area network (GAN), wide area network (WAN), metropolitan area network (MAN), virtual private networks (VPN), or collection of any such communication networks such as an intranet, extranet or the Internet (i.e., a global system of interconnected networks upon which various applications or service run including, for example, the World Wide Web). The client devices generally communicate with the controller/processor over a wireless communication system that can include any suitable wireless communication technology. Non-limiting examples of suitable wireless communication technologies include various cellular-based data communication technologies (e.g., 2G, 3G, 4G, LTE, 5G, GSM, etc.), Wi-Fi wireless data communication, wireless LAN communication technology (e.g., 802.11), Bluetooth wireless data communication, Near Field Communication (NFC) wireless communication, other networks or protocols capable of carrying data, and combinations thereof. In some embodiments, the network may be chosen from the internet, at least one wireless network, at least one cellular communication network, and combinations thereof. As such, the network may include any number of additional devices, such as additional computers, routers, and switches, to facilitate communications. In some embodiments, the network may be, or may include, a single network, and in other embodiments the network may be or include a collection of networks.

The controller/processor may be configured to communicate and share data with the client devices associated with one or more users. Accordingly, the client device may be embodied as any type of device for communicating with the controller/processor and/or other client devices over the network. For example, at least one of the client devices may be embodied as, without limitation, a computer, a desktop computer, a personal computer (PC), a tablet computer, a laptop computer, a notebook computer, a mobile computing device, a smart phone, a cellular telephone, a handset, a messaging device, a work station, a distributed computing system, a multiprocessor system, a processor-based system, and/or any other computing device configured to store and access data, and/or to execute software and related applications consistent with the present disclosure.

A client device may be configured as a mobile device (e.g., smartphone, tablet computer, etc.), although it should be noted that embodiments are not limited to mobile client devices and instead could include, for example, desktop computers and other computing devices. The client device generally includes a computing system. The computing system may include one or more processors, which can include, for example, at least one main processor (e.g., microprocessor, microcontroller, central processing unit, etc.) and optionally also at least one graphics processing unit (GPU) for performing any of various graphics-specific operations including, for example, producing tessellated graphics for display on a display unit (although in some embodiments the main processor may include a GPU and/or an extended instruction set of graphics instructions, or otherwise may perform graphics-specific operations such as in software). For convenience, the one or more processors may be referred to herein collectively as a processor. The processor may be operably connected to a communication infrastructure (e.g., a communications bus, cross-over bar, or network). The processor may be embodied as any type of processor capable of performing the functions described herein. For example, the processor may be embodied as a single or multi-core processor(s), digital signal processor, microcontroller, or other processor or processing/controlling circuit.

The computing system may further include a display interface that forwards graphics, text, sounds, and other data from the communication infrastructure (or from a data buffer) for display on a display unit. The computing system may further include any of various types of input devices such as for interacting with the mobile device, such as a keypad, microphone, camera, as well as other input components, including motion sensors, and the like. In one embodiment, the display unit may include a touch-sensitive display (also known as “touch screens” or “touchscreens”), in addition to, or as an alternative to, physical push-button keyboard or the like. The touch screen may generally display graphics and text, as well as provides a user interface (e.g., but not limited to graphical user interface (GUI)) through which a user may interact with the mobile device, such as accessing and interacting with applications executed on the device.

The computing system may further include a main memory, such as random access memory (RAM), and may also include a secondary memory. The main memory and secondary memory may be embodied as any type of device or devices configured for short-term or long-term storage of data such as, for example, memory devices and circuits, memory cards, hard disk drives, solid-state drives, or other data storage devices. Similarly, the memory may be embodied as any type of volatile or non-volatile memory or data storage capable of performing the functions described herein.

The mobile device may maintain one or more application programs, databases, media, and/or other information in the main and/or secondary memory. The secondary memory may include, for example, a hard disk drive and/or removable storage drive, representing a floppy disk drive, a magnetic tape drive, an optical disk drive, etc. Removable storage drive can read from and/or write to the removable storage unit in any known manner. The removable storage unit may represent a floppy disk, magnetic tape, optical disk, etc. which is read by and written to by the removable storage drive. As will be appreciated, the removable storage unit may include a computer usable storage medium having stored therein computer software and/or data.

In alternative embodiments, the secondary memory may include other similar devices for allowing computer programs or other instructions to be loaded into the computing system. Such devices may include, for example, a removable storage unit and interface. Examples of such may include a program cartridge and cartridge interface (such as that found in video game devices), a removable memory chip (such as an erasable programmable read only memory (EPROM), or programmable read only memory (PROM)) and associated socket, and other removable storage units and interfaces, which allow software and data to be transferred from the removable storage unit to the computing system.

The computing system may further include one or more application programs directly stored thereon. The application program(s) may include any number of different software application programs, each configured to execute a specific task.

The computing system may further include a communications interface. The communications interface may be embodied as any communication circuit, device, or collection thereof, capable of enabling communications between the mobile device external devices (other mobile devices, the cloud-based service, and the external computing system/server). The communications interface may be configured to use any one or more communication technology and associated protocols, as described above, to effectuate such communication. For example, the communications interface may be configured to communicate and exchange data with the server, the external computing system/server, and/or another mobile device via a wireless transmission protocol including, but not limited to, Bluetooth communication, infrared communication, near field communication (NFC), radio-frequency identification (RFID) communication, cellular network communication, versions of IEEE 802.11 transmission protocol standards, and a combination thereof. Examples of communications interface may include a modem, a network interface (such as an Ethernet card), a communications port, a Personal Computer Memory Card International Association (PCMCIA) slot and card, wireless communication circuitry, etc.

Computer programs (also referred to as computer control logic) may be stored in main memory and/or secondary memory or a local database on the mobile device. Computer programs may also be received via the communications interface. Such computer programs, when executed, enable the computing system to perform the features of the present invention, as discussed herein. In particular, the computer programs, including application programs, when executed, enable processor to perform the features of the present invention. Accordingly, such computer programs represent controllers of computer system.

In one embodiment where the invention is implemented primarily in software, the software may be stored in a computer program product and loaded into the computing system using a removable storage drive, hard drive, or communications interface. The control logic (software), when executed by the processor, causes the processor to perform the functions of the invention as described herein. In another embodiment, the invention may be implemented primarily in hardware using, for example, hardware components such as application specific integrated circuits (ASICs). Implementation of the hardware state machine so as to perform the functions described herein will be apparent to persons skilled in the relevant art(s). In yet another embodiment, the invention may be implemented using a combination of both hardware and software.

Similarly, the controller/processor may include one or more processors (e.g., one or more microprocessors, microcontrollers, central processing units, computers, cloud computing systems, digital signal processors, etc.), any of various types of data storage (e.g., RAM, ROM, cloud storage, etc.) such as for storing computer program instructions and/or other types of data, a graphical user interface that produces or interacts with graphical user interfaces through which various entities can interface with the server system (e.g., a graphical user interface presented on a client device), and a communication interface that utilizes one or more communication technologies or protocols to communicate with the client devices and/or other entities via the network.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In alternative embodiments, the disclosed apparatus and methods (e.g., as in any flow charts or logic flows described above) may be implemented as a computer program product for use with a computer system. Such implementation may include a series of computer instructions fixed on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as a tangible, non-transitory semiconductor, magnetic, optical or other memory device, and may be transmitted using any communications technology, such as optical, infrared, RF/microwave, or other transmission technologies over any appropriate medium, e.g., wired (e.g., wire, coaxial cable, fiber optic cable, etc.) or wireless (e.g., through air or space).

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

Computer program logic implementing all or part of the functionality previously described herein may be executed at different times on a single processor (e.g., concurrently) or may be executed at the same or different times on multiple processors and may run under a single operating system process/thread or under different operating system processes/threads. Thus, the term “computer process” refers generally to the execution of a set of computer program instructions regardless of whether different computer processes are executed on the same or different processors and regardless of whether different computer processes run under the same operating system process/thread or different operating system processes/threads. Software systems may be implemented using various architectures such as a monolithic architecture or a microservices architecture.

Importantly, it should be noted that embodiments of the present invention may employ conventional components such as conventional computers (e.g., off-the-shelf PCs, mainframes, microprocessors), conventional programmable logic devices (e.g., off-the shelf FPGAs or PLDs), or conventional hardware components (e.g., off-the-shelf ASICs or discrete hardware components) which, when programmed or configured to perform the non-conventional methods described herein, produce non-conventional devices or systems. Thus, there is nothing conventional about the inventions described herein because even when embodiments are implemented using conventional components, the resulting devices and systems (e.g., the controller and processors discussed herein with reference to the handheld wand and pad embodiments) are necessarily non-conventional because, absent special programming or configuration, the conventional components do not inherently perform the described non-conventional functions.

The activities described and claimed herein provide technological solutions to problems that arise squarely in the realm of technology. These solutions as a whole are not well-understood, routine, or conventional and in any case provide practical applications that transform and improve computers and computer routing systems.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

1. An apparatus comprising at least one sensor system, each sensor system comprising:
at least one optical receiver;
at least one acoustic receiver; and
a plurality of sets of LEDs, wherein each set of LEDs contains a distinct color of LED, and wherein the sensor system is configured to direct light from the LEDs into skin to induce both optical signals that are detected by the at least one optical receiver and photoacoustic signals that are detected by the at least one acoustic receiver to detect subcutaneous structures through a combination of optical and photoacoustic sensing.

2. The apparatus of claim 1, wherein each set of LEDs includes three LEDs.

3. The apparatus of claim 1, wherein the LEDs are arranged around the at least one ultrasonic transducer.

4. The apparatus of claim 1, wherein the plurality of sets of LEDs includes a set of orange LEDs and a set of UV LEDs.

5. The apparatus of claim 1, wherein the plurality of sets of LEDs includes a set of orange LEDs, a set of UV LEDs, and a set of red LEDs.

6. The apparatus of claim 1, wherein the plurality of sets of LEDs includes at least two of:
a set of orange LEDs;
a set of UV LEDs;
a set of red LEDs;
a set of near-infrared LEDs; or
a set of infrared LEDs.

7. The apparatus of claim 1, further comprising a controller configured to control the LEDs and/or process received optical and acoustic signals.

8. The apparatus of claim 1, wherein the at least one optical receiver comprises at least one photodiode.

9. The apparatus of claim 1, wherein the at least one acoustic receiver comprises at least one ultrasonic receiver or transducer.

10. (canceled)

11. The apparatus of claim 1, further comprising a housing containing the at least one sensor system, wherein the housing comprises a handheld wand or a pad.

12. (canceled)

13. The apparatus of claim 1, wherein the at least one sensor system comprises a plurality of sensor systems, optionally wherein the sensor systems are arranged in an array such as a 4×4 array.

14. (canceled)

15. (canceled)

16. (canceled)

17. The apparatus of claim 1, further comprising at least one of an interface to an external controller, an internal power source, or an interface to an external power source.

18. (canceled)

19. (canceled)

20. A system comprising:
an apparatus according to claim 1; and
a controller separate from the apparatus, wherein the apparatus and the controller are configured to communicate over a communication connection.

21. (canceled)

22. (canceled)

23. The system of claim 20, wherein the controller provides power to the apparatus.

24. (canceled)

25. The system of claim 20, wherein the controller is configured to control the LEDs and/or process received optical and acoustic signals.

26. The system of claim 25, wherein the controller is configured to display detected subcutaneous structures on a display in real-time, optionally wherein the displayed subcutaneous structures are color-coded and/or the displayed subcutaneous structures are displayed in 3D and are manipulable in 3D.

27. (canceled)

28. (canceled)

29. A method for detecting subcutaneous structures, the method comprising:
activating at least one LED to direct LED light into skin of a patient;
receiving optical signals returning through the skin in response to the LED light;
receiving photoacoustic signals returning through the skin in response to the LED light; and
processing the optical signals and the photoacoustic signals to detect at least one subcutaneous structure.

30. The method of claim 29, wherein the at least one subcutaneous structure includes at least one of arteries, veins, nerves, hard tissue/bone, foreign objects, etc.

31. The method of claim 29, wherein the at least one LED comprises LEDs of at least two different colors.

32. The method of claim 29, further comprising:
controlling the intensity of the LED light.
